(19) United States
(12) Patent Application Publication
(10) Pub. No.: US 2006/0258844 A1
(43) Pub. Date: Nov. 16, 2006

(54) Algae fluorescence protein dry powder structure and manufacturing method thereof

(75) Inventors: Jiunn-Ming Jeng, Sinjhuang City (TW); Chih-Yi Lu, Jhongpu Township (TW); Wen-Jen Yang, Bade City (TW)

Correspondence Address: LOWE HAUPTMAN BERNER, LLP, 1700 DIAGONAL ROAD, SUITE 300, ALEXANDRIA, VA, 22314, US

(73) Assignee: Zen-U Biotechnology Co., Ltd., Sindian City (TW)

(21) Appl. No.: 11/328,193

(22) Filed: Jan. 10, 2006

(30) Foreign Application Priority Data
May 12, 2005 (TW) 094115360

(51) Int. Cl. C07K 14/405; C12P 19/04

(52) U.S. Cl. 530/350; 536/123

(57) ABSTRACT

This present invention is related to an algae fluorescence protein dry powder structure and the manufacturing method thereof. The algae fluorescence protein dry powder structure includes a substrate and a mixture layer, which is coated around the substrate. The mixture layer consists of polysaccharide and algae fluorescence protein, wherein the weight proportion of the polysaccharide should be smaller than 10%. The method will dry the algae fluorescence protein and then protect the structure of them from damage.

### BACKGROUND OF THE INVENTION

1. Field of the Invention

The present invention relates to an algae fluorescence protein dry powder structure and the manufacturing method thereof, especially to a manufacturing method for drying the algae fluorescence protein without damaging the molecular structure thereof, and especially to an algae fluorescence protein dry powder structure manufactured thereby.

2. Description of the Related Arts

The algae fluorescence protein is one kind of fluorescence pigment abstracted from algae. The source, i.e. the algae, is so crude that it will not cause qualm of damage to human beings, not like general chemical materials. The fluorescence pigment abstracted form algae, such as algae red pigment fluorescence protein or algae blue pigment fluorescence protein, can be applied to food or to medicament, which is a great progress in bioscience.

As known, generic algae fluorescence protein abstracted from algae is generally liquid state, and is not convenient for transportation or carrying, which affects the popularization of this product. To resolve this problem, drying the liquid algae fluorescence protein is a prevalent solution. Conventional drying methods, no more than spraying drying method and cooling drying method, are applied to dry the liquid algae fluorescence protein. By the conventional drying method, the liquid algae fluorescence protein is changed to solid state. However, conventional methods will damage the molecule structure of the algae fluorescence protein and will cause transformation. Accordingly, the dried algae fluorescence protein cannot revert to the original molecule structure that can produce fluorescence effect.

A prior technology is recited to resolve the above-mentioned problem, as disclosed in Japanese unexamined Invention Model publication No. 11-299450. A drying method is brought out. The drying method is to add trehalose into the algae fluorescence protein and then to cooling the liquor for drying. The weight proportion of the trehalose in the liquor should be greater than 10%. The trehalose is served to protect the molecule structure of the algae fluorescence protein from being damaged during the drying process. However, this method costs too much. A new method having simple steps and costing lower is accordingly desired.

### SUMMARY OF THE INVENTION

A major object of the present invention is to provide a manufacturing method of algae fluorescence protein dry powder structure, without damaging the molecular structure of the, for changing the algae fluorescence protein to dry powder structure and for transporting or carrying the algae fluorescence protein conveniently.

According to the above-mentioned object, a manufacturing method of algae fluorescence protein dry powder structure is accordingly provided by the present invention. The manufacturing method includes the following steps: proving a substrate, providing mixed liquid consisting of polysaccharide and algae fluorescence protein, coating the mixed liquid around the substrate for forming mixture layer, and drying the substrate with the mixture layer thereon. After the method said above, the algae fluorescence protein dry powder structure is accordingly obtained.

According to the above-described invention, the coating and drying step can be one of spraying coating method and floating bed coating method.

According to the above-described invention, the substrate can be selected from maltose, lactose, starch or combination of the forgoing.

According to the above-described invention, the composition proportion of the polysaccharide to the total is smaller than 10%.

According to the above-described invention, the weight proportion of the polysaccharide to the total should be smaller than 10%.

According to the above-described invention, in the mixture layer, the weight proportion of the polysaccharide to the algae fluorescence protein is greater than 10%.

According to the above-described invention, an algae fluorescence protein dry powder structure is provided, and the structure includes a substrate completely coated around by a mixture layer, wherein the mixture layer consists of polysaccharide and algae fluorescence protein, and the composition proportion of the polysaccharide to the total is smaller than 10%.

According to the above-mentioned invention, the mixture layer of the algae fluorescence protein dry powder structure completely covers the substrate.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

The present invention will become more apparent from the following detailed description when taken in conjunction with the accompanying drawings, in which:

### BRIEF DESCRIPTION OF THE DRAWINGS

FIG. 1 is a flow chart of a manufacturing method of an algae fluorescence protein dry powder structure in accordance with a preferred embodiment of the present invention; and

FIG. 2 is an illustration of the algae fluorescence protein dry powder structure in accordance with the present invention.

### DESCRIPTION OF PREFERRED EMBODIMENT

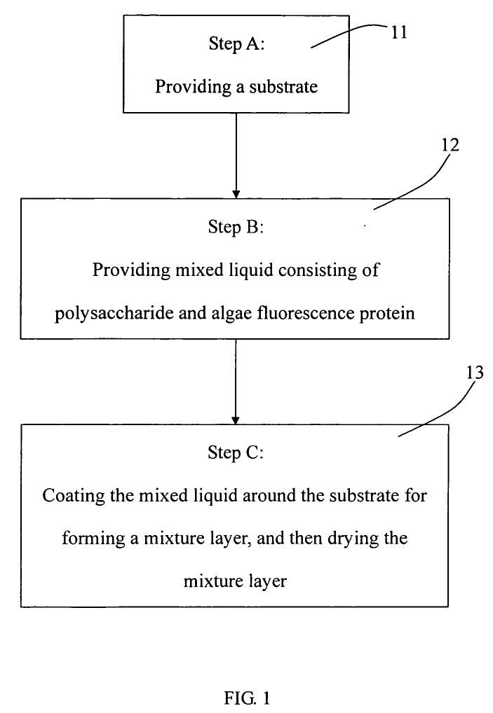

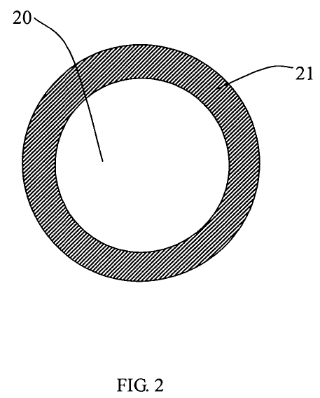

Reference will now be made to FIGS. 1-2 to describe the present invention in detail, wherein, FIG. 1 is a flow chart of a manufacturing method of an algae fluorescence protein dry powder structure in accordance with a preferred embodiment of the present invention; and FIG. 2 is an illustration of the algae fluorescence protein dry powder structure in accordance with the present invention.

The manufacturing method includes the following steps:

step A, providing a substrate 20, illustrated as step 11 in FIG. 1, wherein the substrate 20 can be selected form maltose, lactose, starch or combination of the forgoing, for supporting the molecule structure of the dried algae fluorescence protein, and wherein the step A actually provides a plurality of granule-shaped substrates 20, weight of each substrate 20 being limited in a predetermined range for controlling the holistic proportion;

step B, providing mixed liquid, illustrated as step 12 in FIG. 1, wherein the mixed liquid consists of polysaccharide and algae fluorescence protein, the mixture of the polysaccharide and the algae fluorescence protein is served as protector to the molecule structure of the algae fluorescence protein;

step C, coating the mixed liquid around the substrate 20 for forming a mixture layer 21, and then drying the mixture layer, illustrated as step 13 in FIG. 1, wherein the coating method can be selected form spraying coating method, floating bed coating method, or other similar methods, for adequately coating the mixed liquid around the substrate 20 to obtain mixture layer 21, and wherein it is a better mode that the mixture layer should completely cover the substrate 20.

After the above-mentioned steps, granule-shaped algae fluorescence protein dry powder structure is accordingly obtained. The polysaccharide can be selected form trehalose, alcohol or other similar matters. The composition proportion of the polysaccharide to the total is smaller than 10%. It is a better mode that the weight proportion of the polysaccharide to the total is smaller than 10%, and the weight proportion of the polysaccharide in the mixture layer to the algae fluorescence protein in the mixture layer is greater than 10%, which enables the polysaccharide to protect the structure of the algae fluorescence protein from being damaged during drying process.

Based on the description above, molecular structure of the algae fluorescence protein in the algae fluorescence protein dry powder structure 2 is protected by the polysaccharide on the substrate form being damaged. Because the weight proportion of the polysaccharide is limited smaller than 10%, the algae fluorescence protein dry powder structure 2 can be revert to the original status of the algae fluorescence protein and retain the same fluorescence character as that before being dried, which is proved by the inventor via a series of experiments. It is really proved that the manufacturing method of the present invention can dry the algae fluorescence protein without damaging the molecular structure thereof and can achieve the purpose of transportation and carrying.

Furthermore, although the present invention has been described with reference to particular embodiment, it is not to be construed as being limited thereto. Various alterations and modifications can be made to the embodiments without in any way departing from the scope or spirit of the present invention as defined in the appended claims.

1. An algae fluorescence protein dry powder structure, comprising a substrate completely coated by a mixture layer around an outer surface of the substrate, wherein the mixture layer comprises polysaccharide and algae fluorescence protein, and a composition proportion of the polysaccharide to the total structure is smaller than 10%.

2. The algae fluorescence protein dry powder structure as cited in claim 1, wherein the substrate is selected from maltose, lactose, starch or combination of the forgoing.

3. The algae fluorescence protein dry powder structure as cited in claim 1, wherein the polysaccharide is trehalose.

4. The algae fluorescence protein dry powder structure as cited in claim 1, wherein the weight proportion of the polysaccharide to the total structure is smaller than 10%.

5. The algae fluorescence protein dry powder structure as cited in claim 1, wherein the mixture layer completely covers the substrate.

6. The algae fluorescence protein dry powder structure as cited in claim 1, wherein the proportion of the weight of the polysaccharide to the weight of the algae fluorescence protein is greater than 10%.

7. A process of making an algae fluorescence protein dry powder structure, comprising the steps of:
providing a substrate selected from maltose, lactose, starch or combination of the forgoing;
providing mixed liquid comprising plysaccharide and algae fluorescence protein; and
coating the mixed liquid around the substrate for forming a mixture layer, and then drying the mixture layer, thereby obtaining the algae fluorescence protein dry powder structure.

8. The process of making an algae fluorescence protein dry powder structure as cited in claim 7, wherein the mixture layer is coated around the substrate by a spraying coating method or a floating bed coating method.

9. (canceled)

10. The process of making an algae fluorescence protein dry powder structure as cited in claim 7, wherein the polysaccharide is trehalose.

11. The process of making an algae fluorescence protein dry powder structure as cited in claim 7, wherein a composition proportion of the polysaccharide in the algae fluorescence protein dry powder structure is smaller than 10%.

12. The process of making an algae fluorescence protein dry powder structure as cited in claim 11, wherein a weight proportion of the polysaccharide in the algae fluorescence protein dry powder structure is smaller than 10%.

13. The process of making an algae fluorescence protein dry powder structure as cited in claim 7, wherein a weight proportion of the polysaccharide in the mixed liquid to the algae fluorescence protein in the mixed liquid is greater than 10%.
